(19) United States
(12) Patent Application Publication
(10) Pub. No.: US 2006/0161144 A1
(43) Pub. Date: Jul. 20, 2006

(54) Optical tracking system and associated methods

(76) Inventor: Haizhang Li, Orlando, FL (US)

Correspondence Address: JACQUELINE E. HARTT, PH.D;ALLEN, DYER, DOPPELT, MILBRATH & GILCHRIST, P.A., P.O. BOX 3791, ORLANDO, FL, 32802-3791, US

(21) Appl. No.: 11/301,642

(22) Filed: Dec. 13, 2005

Related U.S. Application Data
(63) Continuation-in-part of application No. 10/743,558, filed on Dec. 22, 2003, now Pat. No. 7,001,377.

(51) Int. Cl. G01P 3/36; A61F 9/008

(52) U.S. Cl. 606/10; 606/5; 356/29

(57) ABSTRACT

An eye-tracking method includes the step of removably affixing a ring member to an eye in surrounding relation to a cornea of the eye. A plurality of first light spots are transmitted onto the ring member, a second light spot is transmitted onto a reflective sector of the ring member, and reflections are detected from the first and the second light spots. By analyzing the reflections, translational and tilting eye movement can be detected. A system for tracking eye movement includes a ring member and a device for removably affixing the ring member to an eye in surrounding relation to a cornea of the eye, such as, for example, by applying a vacuum to the ring. The ring member can comprise concentric inner and outer rings having contrasting colors to each other and to the eye areas adjacent to which they are positionable.

### CROSS-REFERENCE TO RELATED APPLICATION

This application is a continuation-in-part of copending application Ser. No. 10/743,558, entitled “Optical Tracking System and Associated Methods,”filed Dec. 22, 2003, which is commonly owned with the present invention and which is incorporated herein by reference.

### FIELD OF THE INVENTION

The invention relates generally to optical tracking systems, and more particularly to optical systems used with a corneal ablation device.

### BACKGROUND OF THE INVENTION

The use of lasers to erode all or a portion of a workpiece's surface is known in the art. In the field of ophthalmic medicine, modification of corneal curvature is known to be accomplished using ultraviolet or infrared lasers. The procedure has been referred to as “corneal sculpting.”

In such a procedure, application of the treatment laser during unwanted eye movement can degrade the refractive outcome of the surgery. The eye movement or eye positioning is critical since the treatment laser is centered on the patient's theoretical visual axis which, practically speaking, is approximately the center of the patient's pupil. However, this visual axis is difficult to determine due in part to residual eye movement and involuntary eye movement known as saccadic eye movement.

Video-based eye tracking systems automatically recognize and track the position of the eye based upon landmarks present within an image of a human eye. Video-based systems, however, have neither sufficient speed nor accuracy to detect high-speed movement.

Previous disclosure of eye tracking systems and methods has been made in U.S. Pat. Nos. 5,980,513; 6,315,773; and 6,451,008, which are co-owned with the present application, and which are hereby incorporated by reference hereinto. In these patents, an eye treatment laser beam delivery and eye tracking system is provided (FIG. 1). A treatment laser and its projection optics generate laser light along an original beam path (i.e., the optical axis of the system) at an energy level suitable for treating the eye. An optical translator shifts the original beam path in accordance with a specific scanning pattern so that the original beam is shifted onto a resulting beam path that is parallel to the original beam path. An optical angle adjuster changes the resulting beam path's angle relative to the original beam path such that the laser light is incident on the eye.

An eye movement sensor detects measurable amounts of movement of the eye relative to the system's optical axis and then generates error control signals indicative of the movement. The parallel relationship between the eye movement sensor's delivery light path and the treatment laser's resulting beam path is maintained by the optical angle adjuster. In this way, the treatment laser light and the eye movement sensor's light energy are incident on the eye in their parallel relationship.

A portion of the eye movement sensor's light energy is reflected from the eye as reflected energy traveling on a reflected light path back through the optical angle adjuster. The optical receiving arrangement detects the reflected energy and generates the error control signals based on the reflected energy. The optical angle adjuster is responsive to the error control signals to change the treatment laser's resulting beam path and the eye movement sensor's delivery light path in correspondence with one another. In this way, the beam originating from the treatment laser and the light energy originating from the eye movement sensor track along with the eye's movement.

The laser beam delivery and eye tracking system 10 includes treatment laser source 11, projection optics 12, X-Y translation mirror optics 13, beam translation controller 14, dichroic beamsplitter 15, and beam angle adjustment mirror optics 16.

After exiting the projection optics 12, beam 17 impinges on X-Y translation mirror optics 13, where beam 17 is translated or shifted independently along each of two orthogonal translation axes as governed by beam translation controller 14.

The eye tracking portion of system 10 includes eye movement sensor 18, dichroic beamsplitter 15, and beam angle adjustment mirror optics 16. The sensor 18 determines the amount of eye movement and uses same to adjust mirrors 19 and 20 to track along with such eye movement. To do this, sensor 18 first transmits light energy 21, which has been selected to transmit through dichroic beamsplitter 15. At the same time, after undergoing beam translation in accordance with the particular treatment procedure, beam 17 impinges on dichroic beamsplitter 15, which has been selected to reflect beam 17 to the beam angle adjustment mirror optics 16.

Light energy 21 and beam 17 preferably retain their parallel relationship when they are incident on an eye 23. Beam angle adjustment mirror optics 16 consists of independently rotating mirrors 19 and 20 under servo control.

Light energy reflected from the eye 23 travels back through optics 16 and beamsplitter 15 for detection at sensor 18. Sensor 18 determines the amount of eye movement based on the changes in reflection energy 22. Error control signals indicative of the amount of eye movement are fed back by sensor 18 to beam angle adjustment mirror optics 16. The error control signals govern the movement or realignment of mirrors 19 and 20 in an effort to drive the error control signals to zero. In doing this, light energy 21 and beam 17 are moved in correspondence with eye movement while the actual position of beam 17 relative to the center of the pupil is controlled by X-Y translation mirror optics 13.

The light energy should preferably lie outside the visible spectrum so as not to interfere or obstruct a surgeon's view of eye 23, and must be “eye safe” as defined by the American National Standards Institute (ANSI), for example, light energy 21 may be infrared light energy in the 900-nanometer wavelength region.

Sensor 18 may be broken down into a delivery portion and a receiving portion (FIG. 2). Essentially, the delivery portion projects light energy 21 in the form of light spots 24-27 onto a boundary (e.g., iris/pupil boundary 28) on the surface of eye 23. The receiving portion monitors light energy 22 in the form of reflections caused by light spots 24-27.

In use, spots 24 and 26 are focused and positioned on axis 29, while spots 25 and 27 are focused and positioned on axis 30 as shown. Axes 29,30 are orthogonal to one another. Spots 24-27 are focused to be incident on and evenly spaced about iris/pupil boundary 28. The four spots 24-27 are of substantially equal energy and are spaced substantially evenly about and on iris/pupil boundary 28. This placement provides for two-axis motion sensing as described in the above-referenced co-owned patents.

In the above-described tracking system, four probe spots are projected onto the iris/pupil boundary. It is known that the size and shape of the pupil boundary during surgery can vary with illumination levels, fixation lighting, and the emotional state of the patient. When the pupil contracts, the return signal from the pupil decreases relative to the return signal from the iris. This decreases the contrast and results in a degradation of the tracking quality. It is believed that tracking system 10 is effective for eyes having pupils dilated to greater than approximately 5.5 mm. It would be desirable to track an undilated eye and also an eye that, even dilated, has a pupil less than 5.5 mm, or that has an irregular shape.

Alternative tracking targets, such as the iris/sclera boundary, an ink ring boundary, and a circular track affixed to the sclera, were proposed in U.S. Pat. No. 6,315,773, all of which can have drawbacks. The iris/sclera boundary is opaque; so tracking accuracy can be unacceptable for the purposes of laser eye surgery. An ink boundary can be easily washed away by tearing, and can easily be dispersed. A circular tack affixed to the sclera may not be co-centered with the pupil, resulting in a discrepancy between the tracking data and actual pupil movement. In addition, the sclera areas from which the probe beams reflect may have different reflection coefficients, resulting in tracking errors.

It is also known to affix a ring having cross-marks via suction to the eye, wherein the illuminated marks are imaged onto tracking sensors with focusing optics.

This tracking system 10 is effective for pupils dilated to greater than approximately 5.5 mm. It would be desirable to be able to track undilated pupils and those that, even dilated, are less than 5.5 mm, or that have an irregular shape.

### SUMMARY OF THE INVENTION

The present invention is useful for sensing eye position and movement by tracking both eye axis tilt and transverse motion during surgical procedures, such as, for example, photorefractive keratectomy (PRK), phototherapeutic keratectomy (PTK), and laser in situ keratomileusis (LASIK), and can be used on dilated and undilated eyes.

A method of the present invention includes the step of removably affixing a ring member to an eye in surrounding relation to a cornea of the eye. A plurality of incident light spots are transmitted onto the ring member, and reflections are detected from the ring member of the incident light spots. By analyzing the reflections, eye movement can be determined.

A system for tracking eye movement comprises a ring member and means for removably affixing the ring member to an eye in surrounding relation to a cornea of the eye, such as, for example, by applying a vacuum to the ring. Means for transmitting a plurality of incident light spots onto the ring member is provided, as well as means for detecting reflections from the ring member of the incident light spots. Calculational means are also provided for determining eye movement from an analysis of the reflections.

This technique may be used on objects other than corneas.

### BRIEF DESCRIPTION OF THE DRAWINGS

FIG. 1 (prior art) is a block diagram of a laser beam delivery and eye tracking system.

FIG. 2 (prior art) is a block diagram of a prior art eye movement sensor.

FIG. 3 is a side-top perspective view of a vacuum ring connected to a hose.

FIGS. 4A and 4B illustrate two embodiments of the inner face of the ring member.

FIG. 5 is a front view of an eye having the vacuum ring attached thereto with light spots transmitted thereon.

FIG. 6 is a schematic of the optical system of the present invention for tracking eye tilt and transverse movement.

### DETAILED DESCRIPTION OF THE PREFERRED EMBODIMENTS

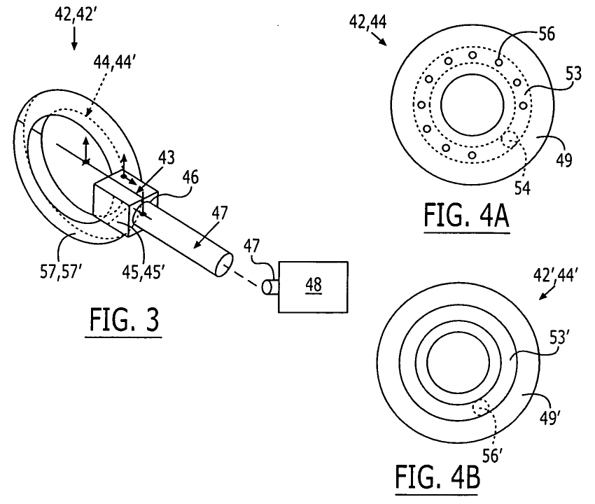

The present invention will now be described with reference to FIGS. 3-6. A system and method for tracking eye tilt and transverse movement comprises a tracking device. The tracking device (FIGS. 3-4B) comprises a ring-member 42,42′. Two embodiments are presented herein, although these are not intended to limit the invention. Both ring members 42,42′ comprise a base 43 affixed to a substantially toroidal ring 44,44′. The base 43 has a channel 45,45′ therethrough extending from a hose aperture 46 at an outside of the base 43.

Affixable to the base 43 via the hose aperture 46 is a hose 47. The hose 47 is in fluid communication with a vacuum source 48. An inner face 49,49′ of the ring 44,44′ is affixable around a cornea 50 and is retainable in place by means of the vacuum source 48 through the hose 47. Preferably a center 51,51′ of the ring 44,44′ is affixable to be substantially coincident with a center 52 of the cornea 50.

In a first embodiment, the ring member 42 (FIG. 4A) comprises a ring 44 that has a toroidal tunnel 53 and a hole 54 from an outside to the tunnel 53, which is in fluid communication with the base's hose aperture 46 through the channel 45. A plurality of apertures 56 extend between the tunnel 53 and the inner face 49 of the ring 44. Vacuum pressure reaches the cornea 50 from the hose 47 through the base channel 45 to the tunnel 53 and thence to the apertures 56.

In a second embodiment, the ring member 42′ (FIG. 4B) comprises a ring 44′ that has a substantially toroidal groove 53′ in its inner face 49′. The groove 53′ is substantially concentric with the ring 44′. A hole 56′ extends from an outside of the ring 44′ to the groove 53′. The groove 53′ is in fluid communication with the base's hose aperture 46 through the channel 45′. Vacuum pressure reaches the cornea 50 from the hose 47 through the base channel 45′ to the groove 53′ and thence to the hole 56′.

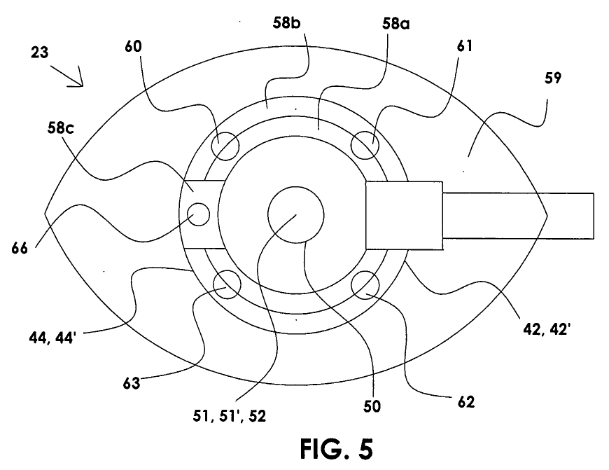

Each ring 44,44′ preferably comprises a color on its outer face 57,57′ that is contrastive with an area of the eye 23 adjacent a location at which the ring member 42,42′ is placed, for improving visibility (FIG. 5). For example, the ring's outer face 57,57′ may comprise an inner ring 58a that has a light color, such as white, for contrast with the cornea 50; an outer ring 58b having a dark color, such as black, provides contrast with the iris 59. The ring's outer face 57,57′ further comprises in a particular embodiment a sector 58c that is highly reflective, for example, mirrored.

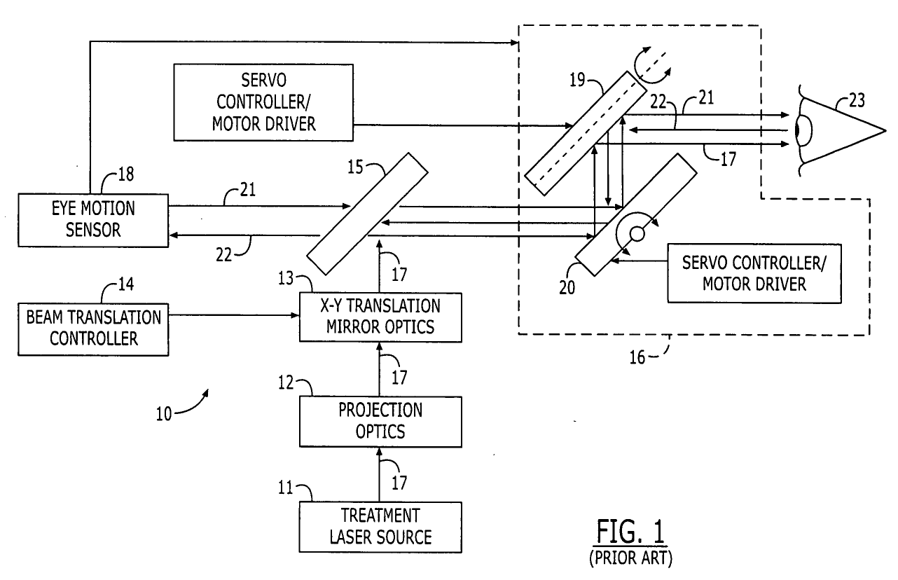

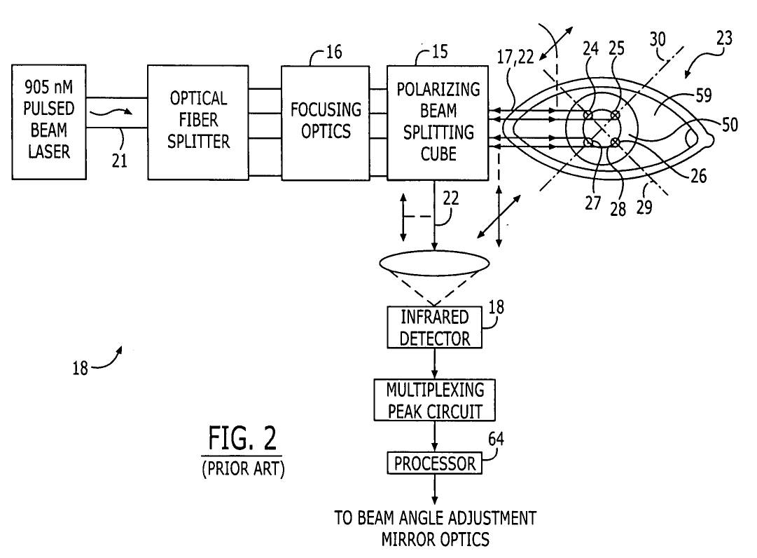

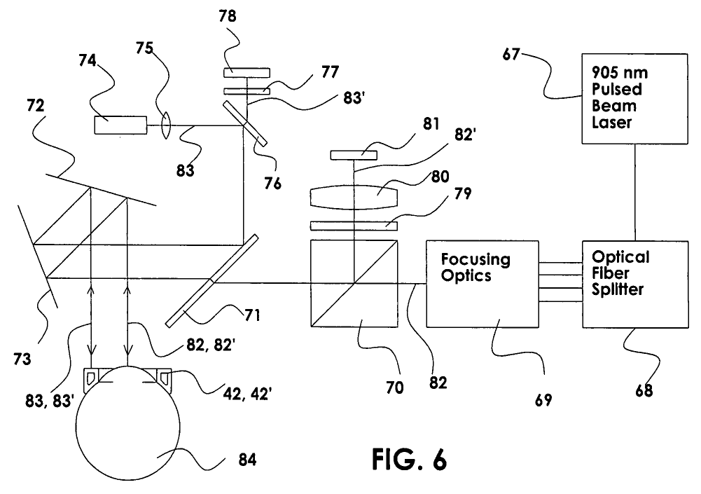

Tracking of eye tilt and transverse movement can be accomplished by means of the optical system illustrated in FIG. 6. Tracking transverse movement using the ring member 42,42′ of the present invention may be achieved in substantially like fashion to that disclosed in the above-referenced '773 patent (FIGS. 1 and 2), wherein a plurality of incident light spots 60-63 are used substantially as the light spots 24-27 of the prior disclosed system 10. Preferably the light spots 60-63 are placed on the boundary of inner ring 58a and outer ring 58b, as illustrated in FIG. 5, using the light emitting and receiving system of FIG. 6.

Light is emitted from a 905-nm pulsed laser source 67, spit by optical fiber splitter 68, and focused by the focusing optics 69. The transmitted beams 82 of spots 60-63 pass through polarizing beamsplitter 70 and beam combiner 71, and are then reflected from an azimuth mirror 72 and elevation mirror 73 onto the ring member's outer face 57,57′. When the eye 23 moves transversely, the four tracking spots 60-63 would move off the boundary of the inner ring 58a and the outer ring 58b, so that the reflected energy level of the spots 60-63 is changed. The beams 82′ from the reflected spots 60-63 are then reflected by the elevation mirror 72 and azimuth mirror 73, pass through beam combiner 71, are reflected from polarizing beamsplitter 70, and are focused by lens 80 onto the eye movement detector 81. A narrow-band filter 79 prevents stray light from entering detector 81.

Eye tilt tracking is accomplished by introducing an additional probe beam 83 from laser light source 74. Probe beam 83 is collimated by lens 75, and is reflected from beamsplitter 76, beam combiner 71, azimuth mirror 73, and elevation mirror 72 onto the outer face 57,57′ of the ring member 42,42′. A preferred location of the spot 66 is indicated on FIG. 5, on the reflective sector 58c. The beam 83′ reflected from spot 66 is reflected by elevation mirror 72, azimuth mirror 73, beam combiner 71, and passes through beamsplitter 76 onto position sensor 78. The filter 77 upstream of the position sensor 78 prevents unwanted light from entering detector 78. When the axis of the eye 23 tilts, reflected beam 83′ deviates from the incident direction, so that the position of reflected beam 83′ on the detector changes accordingly.

Eye tilt and transverse movement are detected using two separate detectors 81 and 78, respectively. Detector 78 can be a commercially available position sensor, such as, but not intended to be limited to, PSM245 Position Sensing Module (On-Trak) or DL-100-7 Position Sensing Photodiode (Silicon Sensor). A typical rise time of the sensors is in the few microsecond range. The sampling speed for tracking eye transverse movement can be 4 kHz, as demonstrated, for example, by the system of the assignee of the present invention, LADAR4000 (Alcon).

Thus the present invention permits the tracking of eye transverse movement and tilt, with a kilohertz sampling speed.

The advantages of the present invention are numerous. Eye tilt and transverse movement are measured quantitatively and used to automatically redirect both the laser delivery and eye tracking portions of the system independent of the laser positioning mechanism. The system operates without interfering with the particular treatment laser or the surgeon performing the eye treatment procedure.

Although the invention has been described relative to specific embodiments thereof, there are numerous variations and modifications that will be readily apparent to those skilled in the art in the light of the above teachings. It is therefore to be understood that, within the scope of the appended claims, the invention may be practiced other than as specifically described.

What is claimed is:

1. A method for tracking eye movement comprising the steps of:
removably affixing a ring member to an eye in surrounding relation to a cornea of the eye, the ring member comprising an inner ring having a first color on an outer face thereof and an outer ring having a second color on the outer face, the inner and the outer rings substantially concentric, the ring member further comprising a reflective sector on the outer face;
transmitting a plurality of first light spots onto the ring member;
detecting reflections from the ring member of the incident light spots;
transmitting a second light spot onto the reflective sector;
determining translational eye movement from an analysis of reflections received from the first light spots; and
determining a tilting eye movement from an analysis of a reflection received from the second light spot.

2. The method recited in claim 1, wherein the first color comprises a light color contrastive with a pupil of the eye and the second color comprises a dark color contrastive with a sclera of the eye.

3. The method recited in claim 1, wherein the reflective sector comprises a mirrored surface.

4. The method recited in claim 1, wherein the affixing step comprises applying a vacuum to at least a portion of the ring member along an interface between the ring member and the eye.

5. The method recited in claim 1, wherein the step of transmitting a plurality of first light spots comprises transmitting four light spots spaced substantially evenly about the ring member.

6. The method recited in claim 5, wherein step of transmitting four light spots comprises directing the four light spots onto a boundary between the inner and the outer ring.

7. The method recited in claim 5, wherein first color is white and the second color is black.

8. The method recited in claim 1, wherein a center of the ring member is substantially coincident with a center of the cornea.

9. The method recited in claim 1, wherein the ring member comprises:
a substantially toroidal ring having a toroidal tunnel and a hole from an outside to the tunnel;
a plurality of apertures extending between the tunnel and an inner face of the ring;
a base affixed to the ring, the base having a channel therethrough extending from a hose aperture at an outside of the base to the hole; and
wherein the affixing step comprises placing the ring inner face around the cornea, connecting a hose to the hose aperture, the hose in fluid communication with a vacuum source, and activating the vacuum source.

10. The method recited in claim 1, wherein the ring member comprises:
a substantially toroidal ring having a substantially toroidal groove in an inner face thereof, the groove substantially concentric with the ring, and a hole from an outside to the groove;
a base affixed to the ring, the base having a channel therethrough extending from a hose aperture at an outside of the base to the hole; and
wherein the affixing step comprises placing the ring inner face around the cornea, connecting a hose to the hose aperture, the hose in fluid communication with a vacuum source, and activating the vacuum source.

11. A system for tracking eye movement comprising:
a ring member comprising an inner ring having a first color on an outer face thereof and an outer ring having a second color on the outer face, the inner and the outer rings substantially concentric, the ring member further comprising a reflective sector on the outer face;
means for removably affixing the ring member to an eye in surrounding relation to a cornea of the eye;
means for transmitting a plurality of first light spots onto the ring member;
means for detecting reflections from the first light spots;
means for determining translational eye movement from an analysis of the first light spot reflections;
means for transmitting a second light spot onto the reflective sector;
means for detecting a reflection from the second light spot; and
means for determining a tilting eye movement from an analysis of the second light spot reflection.

12. The system recited in claim 11, wherein the affixing means comprises a vacuum source in fluid communication with at least a portion of the ring member along an inner face positionable in contact with the eye.

13. The system recited in claim 12, wherein the vacuum source comprises a hose having a first end in fluid communication with a depression in an inner face of the ring member.

14. The system recited in claim 13, wherein the depression comprises a substantially toroidal groove concentric with the ring member.

15. The system recited in claim 11, wherein the first color comprises a light color and the second color comprises a dark color.

16. The system recited in claim 15, wherein the first color is white and the second color is black.

17. The system recited in claim 11, wherein the ring member comprises:
a substantially toroidal ring having a toroidal tunnel and a hole from an outside to the tunnel;
a plurality of apertures extending between the tunnel and an inner face of the ring;
a base affixed to the ring, the base having a channel therethrough extending from a hose aperture at an outside of the base to the hole; and
wherein the affixing means comprises a hose connected to the hose aperture, the hose in fluid communication with a vacuum source, the ring inner face affixable around the cornea and retainable in place by means of a vacuum applied to the ring apertures in contact with the eye.

18. The system recited in claim 11, wherein the ring member comprises:
a substantially toroidal ring having a substantially toroidal groove in an inner face thereof, the groove substantially concentric with the ring, and a hole from an outside to the groove;
a base affixed to the ring, the base having a channel therethrough extending from a hose aperture at an outside of the base to the hole; and
wherein the affixing means comprises a hose connected to the hose aperture, the hose in fluid communication with a vacuum source, the ring inner face affixable around the cornea and retainable in place by means of a vacuum applied to the ring apertures in contact with the eye.
